(19) United States
(12) Patent Application Publication
(10) Pub. No.: US 2019/0264153 A1
(43) Pub. Date: Aug. 29, 2019

(54) BIOREACTOR CONSOLE

(71) Applicant: Juno Therapeutics, Inc., Seattle, WA (US)

(72) Inventors: Chris Duncan VALBURG, Redmond, WA (US); Brian CHRISTIN, Seattle, WA (US); Rachel LOWER, Seattle, WA (US); Pascal BEAUCHESNE, Seattle, WA (US); Dustin John TROIA, Bothell, WA (US)

(73) Assignee: Juno Therapeutics, Inc., Seattle, WA (US)

(21) Appl. No.: 16/319,799

(22) PCT Filed: Jul. 28, 2017
(86) PCT No.: PCT/US2017/044539

§ 371 Date: Jan. 22, 2019

Related U.S. Application Data
(60) Provisional application No. 62/368,915, filed on Jul. 29, 2016.

(51) Int. Cl. C12M 3/00; C12M 1/00; C12M 3/06

(52) U.S. Cl. CPC C12M 23/48; C12M 23/40; C12M 27/16; C12M 23/52; C12M 23/46

(57) ABSTRACT

The present disclosure is directed to bioreactor consoles that provide a stable structure with built-in bioreactor component management. The bioreactor consoles disclosed herein can include one or more vertical platforms with notches and/or a recessed platform that can provide the desirable flexibility to easily connect and disconnect various tubes, cables, and components of the bioreactor system as well as organize which tubes and cables connect to which component of a bioreactor on the vertical platforms.

### REFERENCE TO RELATED APPLICATION

This application claims priority to U.S. Provisional Application No. 62/368,915, filed Jul. 29, 2016, the entire contents of which are incorporated herein by reference.

### FIELD OF THE DISCLOSURE

This disclosure relates in some aspects to consoles, including consoles for bioreactors. More particularly, this disclosure relates in certain aspects to bioreactor consoles that provide a stable structure with built-in bioreactor component management.

### BACKGROUND

Bioreactors can include several components with multiple connections. These components can include, for example, one or more of a reactor, a rocking platform optionally with a lid, one or more various containers including cell containers, such as a cellbag, input containers, a pump module, a gas module, pH/DO devices/controllers, and a waste container. In addition, such components can include multiple fluid lines connecting one or more such components to each other and/or to fluid supply connections as well as power cords and data cables. In operation, it is often a challenge to keep these components and fluid lines uncontaminated and organized. This is especially the case when changing lines and connections during reaction runs and bioreactor modes.

### SUMMARY

Certain available platforms and areas for placing, housing, or operating bioreactors (such as tables, benches, e.g., a typical lab bench) can result in a mess of tubing and/or cables, such as those that become tangled, cluttered, difficult to separate or discern, and can increase various risk. Provided are consoles, such as bioreactor consoles, which address such issues. In some aspects, provided are consoles with a stable structure, typically with built-in bioreactor component management. In some embodiments, the bioreactor consoles can organize the various bioreactor components on one or more vertical platforms, which in some aspects are designed to allow cables/tubes to be easily connected and disconnected from the various bioreactor components on the vertical platforms. Additionally, in some embodiments, bioreactor consoles as described herein are advantageously designed to include features that maintain open fluid lines and/or prevent unwanted kinks or damage to fluid lines or cables. As such, the bioreactor consoles in some aspects can reduce the risk of contamination and/or loss of product; reduce the risk of accidentally manipulating the wrong tube/cable; reduce the risk of mixing up which bioreactor or component associated therewith belongs to which patient/sample; and/or reduce the manufacturing footprint of the bioreactors.

Some embodiments include a console containing a first platform including a first top surface and a first front surface. In some aspects of such embodiments, the first front surface includes one or more notches. In some aspects, such a console further includes a second platform, which may include a second top surface and/or a second front surface. In some aspects, the second platform is located below the first platform and/or a portion of the second front surface is recessed relative to at least a portion of the first front surface. In some aspects, a third platform is located below the second platform. In some aspects, a plurality of legs supporting the first platform, the second platform, and/or the third platform.

Some embodiments include a bioreactor system containing a bioreactor rocker supported on a first platform, wherein in some aspects the first platform includes a first top surface and a first front surface including one or more notches. In such aspects, such a system further includes a bioreactor pump module supported on a second platform located below the first platform, wherein in some aspects the second platform includes a second top surface and a second front surface, and wherein in some aspects a portion of the second front surface is recessed relative to at least a portion of the front surface. In some aspects, such a system further includes a third platform located below the second platform. In some aspects, such a system further includes a plurality of legs supporting the first platform, the second platform, and/or the third platform.

Some embodiments include a bioreactor system containing a bioreactor rocker supported on a first platform. In some aspects of such embodiments, the first platform includes a first top surface and a first front surface including one or more notches. In some aspects, such a system further includes a bioreactor pump module, which may be supported on a second platform located below the first platform. In some aspects, the second platform includes a second top surface and a second front surface. In some aspects, the second front surface is recessed relative to at least a portion of the first front surface. In some aspects, such a system further includes a bioreactor gas module supported on the second platform. In some aspects, such a system further includes a waste container supported on a third platform located below the second platform. In some aspects, such a system further includes a plurality of legs supporting the first platform, the second platform, and/or the third platform. In some aspects, the system further includes an IV pole mounted on at least one leg of the plurality of legs.

In any embodiment, the bioreactor system in some aspects can include a waste container supported on the third platform. In any embodiment, in some aspects, the bioreactor system can include a bioreactor gas module supported on the second platform. In any embodiment, in some aspects, the first platform can extend, or does extend, no more than 12 inches from any side of the bioreactor rocker. In any embodiment, in some aspects, the bioreactor console can include an accessory holder mounted on at least one leg of the plurality of legs. In any embodiment, in some aspects, the bioreactor console can include an IV pole mounted on at least one leg of the plurality of legs. In any embodiment, in some aspects, the bioreactor console can include four legs and the accessory holders are mounted on one or more or at least two of the four legs. In any embodiment, in some aspects, the first front surface can include three notches. In any embodiment, in some aspects, one or more of the one or more notches can be semicircular. In any embodiment, in some aspects, the first platform can include a first back surface, which the first back surface may in some aspects include one or more notches. In any embodiment, in some aspects, the diameter of the notches can be 1 to 6 inches or between about 1 and about 6 inches. In any embodiment, in some aspects, the second front surface can be recessed to be substantially even vertically or plumb with at least a portion of the one or more notches. In any embodiment, in some aspects, the first top surface can be between 2 and 4 feet from a bottom surface of at least one leg of the plurality of legs. In any embodiment, in some aspects, the first front surface can be between 20 and 40 inches long or between about 20 and about 40 inches long. In any embodiment, in some aspects, the first platform can include a first side surface that is between 25 and 45 inches long or between about 25 and about 45 inches long. In any embodiment, in some aspects, the first, second, and third platforms can be made out of stainless steel. In any embodiment, in some aspects, each leg of the plurality of legs can include a vibration dampening foot. In any embodiment, in some aspects, the vibration dampening foot can be adjustable to allow levelling of the bioreactor console. In any embodiment, in some aspects, the first platform can include a first bottom surface and the second top surface can be 6 to 12 inches or between about 6 to about 12 inches below the first bottom surface. In any embodiment, in some aspects, the second platform can include a second bottom surface and the third top surface can be 4 to 10 inches or between about 4 to about 10 inches below the second bottom surface. In any embodiment, in some aspects, the first top surface can include a plurality of tubing clips positioned to guide a plurality of tubes to the one or more notches. In any embodiment, in some aspects, the first top surface can include a plurality of tubing clips and a plurality of color coded markers positioned to guide a tube to each of the plurality of notches.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Although often referred to as a “bioreactor console”, the consoles disclosed herein are not limited to bioreactors in general, but can be used for a variety of purposes known in the art. Additional advantages will be readily apparent to those skilled in the art from the following detailed description. The examples and descriptions herein are to be regarded as illustrative in nature and not restrictive.

### BRIEF DESCRIPTION OF THE DRAWINGS

Exemplary embodiments are described with reference to the accompanying figures, in which:

FIG. 1A illustrates an example of a front view of a bioreactor console disclosed herein.

FIG. 1B illustrates an example of a side view of a bioreactor console disclosed herein.

FIG. 1C illustrates an example of a top view of a first platform of a bioreactor console disclosed herein.

FIG. 1D illustrates an example of a top view of a second platform of a bioreactor console disclosed herein.

FIG. 1E illustrates an example of a top view of a third platform of a bioreactor console disclosed herein.

FIG. 2 illustrates an example of a front view of a bioreactor console with a bioreactor and associated components disclosed herein.

FIG. 3A illustrates a first example of an IV pole of a bioreactor console disclosed herein.

FIG. 3B illustrates a second example of an IV pole of a bioreactor console disclosed herein.

FIG. 3C illustrates a third example of an IV pole of a bioreactor console disclosed herein.

FIG. 4A illustrates a second example of a front view of a bioreactor console with a bioreactor and associated components disclosed herein.

FIG. 4B illustrates a second example of a side view of a bioreactor console with a bioreactor and associated components disclosed herein.

In the Figures, like reference numbers correspond to like components unless otherwise stated.

### DETAILED DESCRIPTION

In some cases, a standard lab bench is used to support a lab's bioreactors and their associated components. However, on a standard lab bench, these components sit side-by-side on the lab bench taking up a large amount of cleanroom space, which is very expensive. Besides taking up cleanroom space, the fluid lines and cables connecting the various bioreactor components to each other can clutter the workspace and become disorganized as to what fluid line or cable belongs to what bioreactor or component. Such a mix-up can result in unwanted contamination and loss of product. Additionally, when fluid lines and cables are not kept separated and organized, kinks or other damage may occur, disrupting the fluid flow and potentially resulting in contamination leaks. There is a need for improved consoles, such as those for improved safety and organization.

In some embodiments, a bioreactor console provides a stable structure with built-in bioreactor component management and/or management of component parts used in conjunction with the bioreactor and/or components and/or parts or units associated with such bioreactors and/or used in a bioprocess, such as components of a closed bioprocessing system. Bioreactors and bioprocessing system components often have tube arrangements that in some aspects have the ability to be connected and/or disconnected, such as easily connected and/or disconnected, between or during runs; such abilities can be important to various aspects of the process. This connection and disconnection generally is preferably performed in such a way as to maintain sterility and to prevent contamination, reduce user error risk, and/or avoid prohibitive cost or time or labor requirements. For example, bioreactors often are used in connection with disposable tubing and other components that can be connected/disconnected between each run and/or when the mode of the bioreactor is changed. With available methods and systems, in which bioreactors sit on benches, such as lab benches, e.g., side-by-side on a standard lab bench, which can create a cluster of tubing and cables, there may be an increased risk of error or other risk. In some cases, an operator may have trouble identifying and manipulating the sought after tube or cable at a given step in the process, which may mean a greater risk of error associated with such step. For example, there may be a greater risk of user error in discerning, or making the correct call about, which tube should be connected to which component of the bioreactor at a particular step in the process, and/or in maintaining sample and/or patient identity during the process, for example, due to an increased risk of mistaking one or more tubes associated with one bioreactor with one or more tubes of another bioreactor, particularly when the two are in close proximity. Such risks can result in unwanted or prohibitive contamination, safety risk, treatment failures, sample or product loss, failed manufacturing runs, and/or increases in manufacturing costs, resource expenditure, and required resources, and/or loss of time and efficiency, and variability among different operators and/or different manufacturing sites running the same or similar process, which in some aspects can result in loss of uniformity, reliability, reproducibility, and/or predictability in the output composition or product, such as therapeutic product, which may lead to safety and other risks.

In contrast to standard lab benches, tables, hoods, and other available housing articles, the consoles described herein in some aspects can reduce one or more of the above risks, e.g., by providing accessible and organized built-in component and part management, such as built-in bioreactor and/or bioreactor component management. The consoles described herein are not necessarily only designed for bioreactors. Instead, the consoles can be used as a workstation for a wide variety of other purposes. For example, the consoles can be used for the transfer or dispensation of media using the IV poles to hold various components (e.g., media bags, etc.), for the bead removal step, for adding media to the culture bag, for holding additional processing equipment such as a Sepax® or Dynamag® devices, or for use as a sampling station during culture.

As described in detail below, the bioreactor consoles in some aspects can include vertical platforms with notches in a platform and/or a recessed platform that can provide the desirable flexibility to easily connect and disconnect various tubes, cables, and components of the bioreactor system as well as organize which tubes and cables connect to which bioreactor component on the vertical platforms. Such bioreactor component management can help minimize the manufacturing footprint of a bioreactor system. In addition, the bioreactor consoles can be ergonomically designed for a bioreactor user.

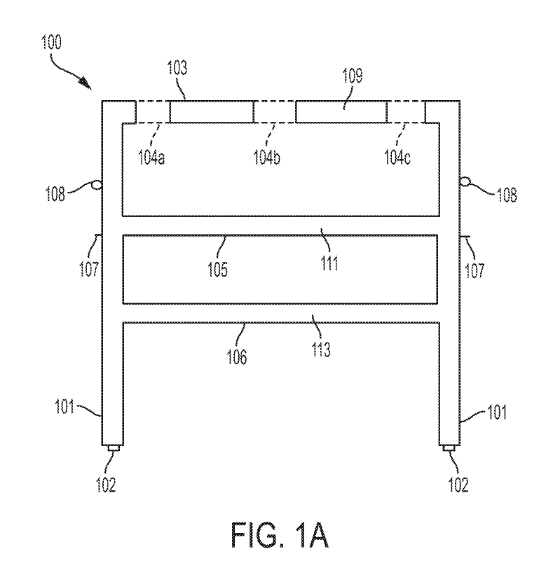

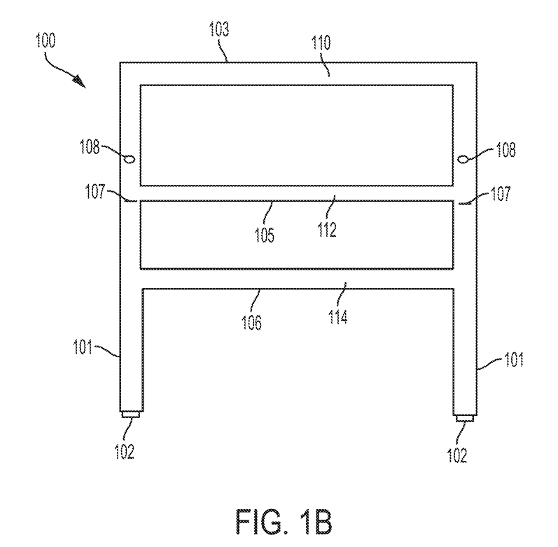

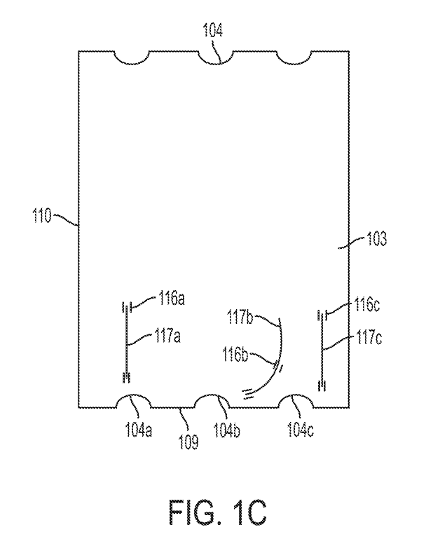

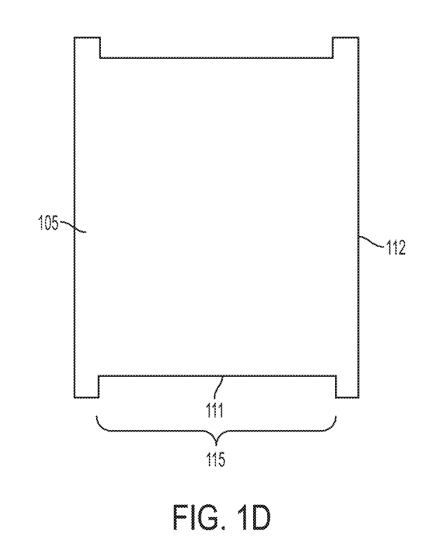

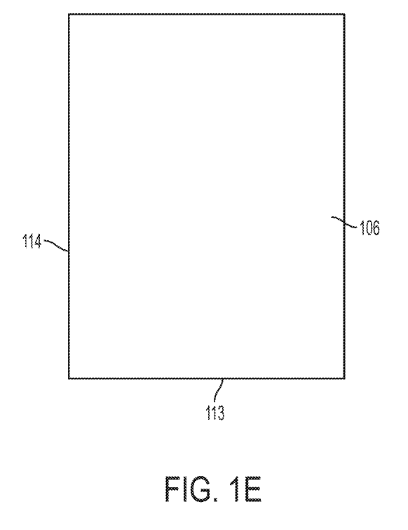

The consoles described herein in some embodiments can be used as a structure to support at least one bioreactor and any associated components. FIG. 1A illustrates an example of a front view of bioreactor console 100. In some embodiments, the front view and the back view can be the same. Bioreactor console 100 in some aspects can have a plurality of legs 101 and platform 103. The plurality of legs in some embodiments can support the platforms of the bioreactor console. For example, legs 101 in some aspects can support platform 103, platform 105, and platform 106 of bioreactor console 100. In some embodiments, the platforms can include the portion of the legs that are supporting the platforms, as shown in FIGS. 1C-1E.

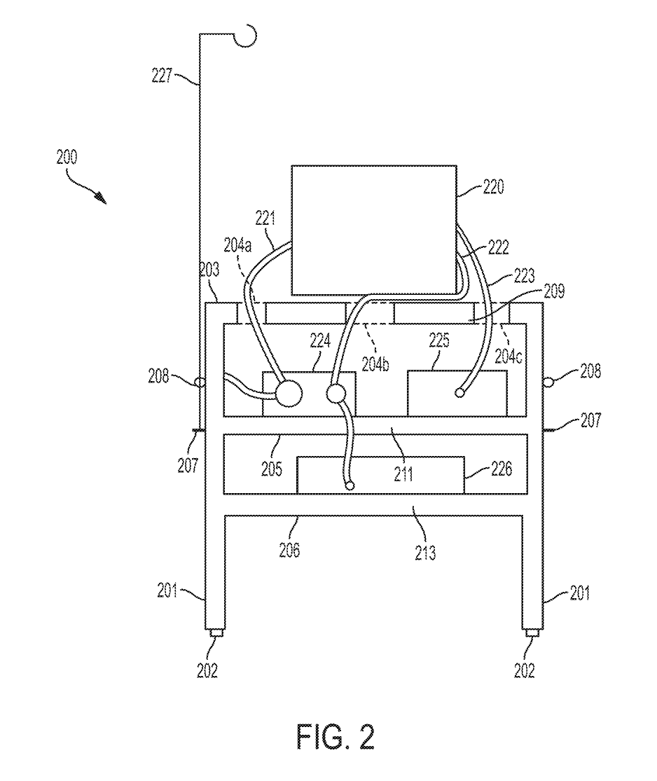

A reactor 220 of the bioreactor in some embodiments can be placed on a top surface of platform 203 as shown in FIG. 2. Examples of bioreactors include, but are not limited to, the bioreactors made by GE (XURI Bioreactor including a dual bag XURI Bioreactor), Applikon, Sartorius, Finesse, Pall, and other bioreactor manufacturing companies, cell therapy manufacturing companies, and biotechnology companies. In some embodiments, the reactor can be a rocker-style reactor (e.g., a rocking platform optionally with a lid) or a continuous stirred tank reactor. In some embodiments, the reactor component of the bioreactor (e.g., a rocker of the bioreactor) can be supported on the top surface of platform 130. The top surface of the top platform can be between about 2-4, 2.5-3.5, or 3 feet from a bottom surface (this can include the foot of the leg) of at least one leg of the bioreactor console.

Besides platform 103, bioreactor console 100 can include a plurality of platforms. For example, FIG. 1A includes platform 105 located below platform 103 and platform 106 located below platform 105. In some embodiments, bioreactor console can include a fourth platform below platform 106. The platforms can be solid and/or perforated. The platforms can support various components of a bioreactor, such as one or more reactors, one or more pump modules, one or more gas modules, and/or one or more waste containers. As shown in FIG. 2, bioreactor pump module 224 and bioreactor gas module 225 can be supported on platform 205. However, a pump module or a gas module can be on any other platform of the bioreactor console. In some embodiments, the reactor component may be combined with the pump module and/or the gas module. For example, the gas module or pump module may be integrated into the reactor. In some embodiments, the gas module can be a CBCU unit which can include a gas mixer, a pH monitor, and/or a DO monitor. In some embodiments, any platform can include a pH and/or DO monitoring device/controller. For example, a pH and/or DO monitoring device/controller can be on the second platform.

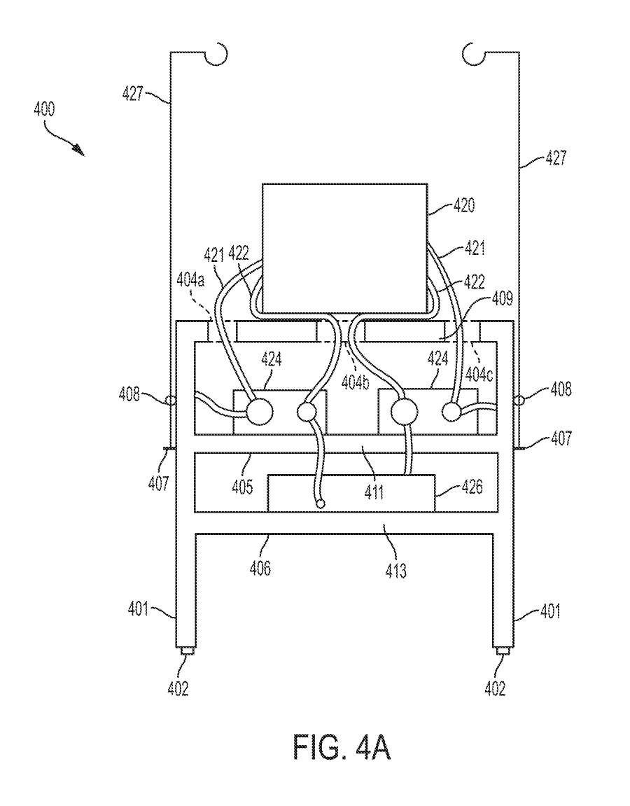

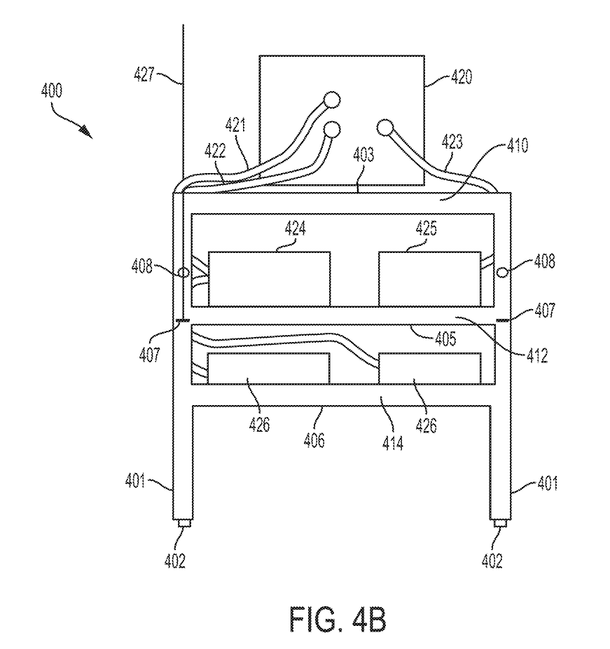

In some embodiments, a platform can include multiple pump modules and/or multiple gas modules. For example, as shown in FIGS. 4A and 4B, platform 405 includes two pump modules 424 and two gas modules 425 (the second gas module is behind (not shown) the gas module shown in FIG. 4B). These pump modules have multiple feed tubes 421, multiple waste tubes 422, and multiple gas tubes 423.

In some embodiments, a pump module can move fluid into and out of the reactor of the bioreactor. For example, a bioreactor might include components such as at least one feed tube and/or at least one waste tube. The pump module in some aspects can supply the reactor with a reactor feed (e.g., media and/or a cell suspension) from a feed source through a feed tube and the pump module can remove waste (e.g. perfusate) from the reactor through a waste tube. In some embodiments, multiple pump modules 424 can supply the reactor 420 with a reactor feed from multiple feed sources (e.g., multiple media bags) through multiple feed tubes 421 and the multiple pump modules 424 can remove waste from the reactor through multiple waste tubes 422 to multiple waste containers 426 as shown in FIGS. 4A and 4B. The gas module in some aspects can move gas to the reactor. For example, a bioreactor might include a component such as a gas tube. The gas module can supply the reactor with gas (e.g., CO₂, oxygen, nitrogen, air, or mixtures thereof) from a gas source through a gas tube during reactor operation. As shown in FIG. 2, waste container 226 in some aspects can be supported on platform 206. However, a waste container can be on any other platform of the bioreactor console. In some embodiments, platform 206 includes multiple waste containers. For example, FIG. 4B shows two waste containers 426 on platform 406. The waste container can receive waste from the reactor. For example, the waste container can receive waste (e.g., perfusate during perfusion mode of the bioreactor) from the reactor through the waste tube.

As described above, bioreactor systems can optionally comprise components including various fluid lines (e.g., tubing) and cables for operation. For example, a bioreactor may include feed tubing, waste tubing, gas tubing, data collection cables, and/or power cables among others. In some applications, a bioreactor can be used to expand cells. During cell expansion, a bioreactor may switch between a static and perfusion mode depending on the cell concentration. For each bioreactor mode, a different feed source may be connected to the bioreactor. Accordingly, the feed tubing for a given bioreactor needs to be easily identifiable and accessible in order to be replaced to avoid any contamination issues. On lab benches, these tubes and cables often clutter the workspace, not only decreasing the amount of space available to place other bioreactors (and corresponding components), but increasing the risk of manipulating the wrong tube or connecting/disconnecting the wrong tube to the wrong component. As such, notches can be used for bioreactor component management. In some embodiments, a surface of a platform can include one or more notches. The platforms of the bioreactor console can have a front surface, back surface, and side surfaces. In some embodiments, there can be one or more notches. In some embodiments, there can be two notches, three notches, four notches, or five notches in a surface of a platform. FIG. 1's front surface 109 includes three notches (104a, 104b, and 104c). In some embodiments, a notch is used for a single tube or cable (or no tube or cable). By having only a single tube or cable per notch, it can be easier to keep the various tubes/cables separated from other tubes/cables. In other embodiments, a notch can be used for multiple tubes and/or cables. For example as shown in FIG. 4A, notch 404b can be used for both waste tubes 422, whereas notch 404a and notch 404c each have a single feed tube 421. As shown in FIG. 2, in some aspects, notches 204a-c can be used for a single tube. Specifically, notch 204a can be used for feed tube 221, notch 204b can be used for waste tube 222, and notch 204c can be used for gas tube 223. In some embodiments, one or multiple notches in the back of the console can be used for at least some of the tubes. For example, FIG. 4B illustrates gas tube 423 (other gas tube not shown) using a notch in the back of the console instead of the front. The notches in some embodiments can allow tubes and/or cables to pass between the reactor and other components of the bioreactor system such as a pump module, gas module, waste container, power outlet, and/or computer, among others. In some embodiments, gas feed lines (e.g., CO2, oxygen, nitrogen, and/or air tubes) can run from a gas source(s) through one or more notches in the back surface of the platform and into the gas module. In addition, power cables and/or data collection cables in some embodiments can run from a reactor through one or more notches in the back surface of a platform and to a power outlet and/or computer.

The notches can come in a variety of shapes and sizes. In some embodiments, the notches can be circular in nature (e.g., a portion of a circle), semicircular, square, rectangular, triangular, or other various geometries. A notch can have a notch depth of about 0.5 to about 5, about 1 to about 3, about 1.5 to about 2, about 1.5, about 2, or about 3 inches into the platform. In addition, a notch can have a notch width of about 0.5 to about 6, about 1 to about 6, about 1.5 to about 5, about 2 to about 4, about 2.5 to about 3.5, or about 3 inches along the surface of the platform. Furthermore, the length between two adjacent notches can be about 3 to about 12, about 4 to about 10, about 5 to about 8, about 6 to about 7, or about 6.5 inches along the surface of the platform. In addition, the length between a notch and an end of the surface (e.g., front surface) of the platform can be about 0.5 to about 6, about 1 to about 3, about 1 to about 2, about 2, or about 1.5 inches. FIG. 1C is a top view of platform 103 that has notches 104 that are shaped as a semicircle in front surface 109. Notches that are shaped as a semicircle can have a diameter of about 0.5 to about 6, about 1 to about 6, about 1.5 to about 5, about 2 to about 4, about 2.5 to about 3.5, or about 3 inches. In addition, the notches do not have to have the same shape. Some notches can have one shape while other notches have a different shape. In some specific embodiments, the notches are not holes through the platform. In such embodiments, the notches are notches in an edge of the front surface of the platform as shown in FIG. 1C, for example.

As stated above, various bioreactors often may be used in association with preassembled and/or disposable components including tube arrangements that need to be easily connected and disconnected. Accordingly, the notches of the bioreactor consoles disclosed herein in some aspects can provide the desirable flexibility to connect and disconnect various tubes, cables, and components of the bioreactor system without having to thread the tubes, cables, and components through holes which can compromise sterility and efficiency (and which might not even be possible if the components are larger than the holes). Furthermore, the notches can, in some aspects, provide a secure and dedicated space for the tubes and/or cables as to prevent accidentally brushing up against or catching the tubes and/or cables if a user works around the bioreactor console. Accordingly, the notches, in some aspects, can prevent any of the tubes and/or cables from sticking off the general perimeter of the console. In addition, the notches in some embodiments can allow for the console to be pushed up against a wall or other surface without any of the tubes and/or cables being pinched by the wall or other object. For example, a top view of the perimeter of the bioreactor console with a bioreactor and associated components would substantially resemble the perimeter of FIG. 1E. There may be one or more tubing lines and/or cables protruding or extending from the general perimeter of the console, such as those that are either connecting to or connecting from other devices (i.e., a computer or gas source) that are not on the bioreactor console itself. In addition, an accessory holder and any corresponding accessory (e.g., an IV stand) may extend from or protrude from the general perimeter of the console, such as in certain aspects described in detail below.

The top surface of a platform can also include a plurality of guiding components positioned to guide a plurality of tubes to the one or more notches. Such guiding components can include, but are not limited to, tubing clips and/or color coded markers. Tubing clips can in some aspects hold tubes in place and/or keep the various tubing lines organized and/or provide for easier, less error-prone and/or quicker identification. In addition, the tubing clips can allow the tubes to “snap” in and out easily and maintain slack leading to the reactor (e.g., rocker platform). The color coded markers can provide visual aids for tube/cable installation. An example of a color coded marker includes colored tape such as vinyl colored tape. For example, FIG. 1C includes tubing clips 116 and color coded markers 117. For instance, tubing clips 116a can help guide a feed tube to notch 104a, tubing clips 116b can help guide a waste tube to notch 104b, and tubing clips 116c can help guide a gas tube to notch 104c. In addition, the color coded markers can be a variety of colors. Preferably, each color coded marker is a different color. For example, color coded marker 117a can be yellow, color coded marker 117b can be red, and color coded marker 117c can be green.

A platform can also include a back surface opposite the front surface. The back surface can be identical to the front surface or it can be different. For example, as shown in FIG. 1C, front surface 109 is identical to back surface opposite front surface 109. Besides a front and back surface, a platform can also have side surfaces. Side surfaces can be identical to each other or can be different. In addition, side surfaces can be identical to the front and/or back surface. For example, FIG. 1B illustrates an example of a side view of a bioreactor console disclosed herein. In some embodiments, both side views of the bioreactor console can be the same. Bioreactor console 100 can have a side surface 110 of platform 103 that does not include notches. Platform 105 can have front surface 111 and side surface 112 and platform 106 can have front surface 113 and side surface 114.

In some embodiments, a portion of a surface of a platform can be recessed relative to at least a portion of a surface of another platform or to at least a portion of the same surface of the same platform. For example, portion 115 of front surface 111 of platform 105 can in some aspects be recessed relative to at least a portion of front surface 109 of platform 103 as shown in FIGS. 1C-1D. In addition, portion 115 of front surface 111 of platform 105 can be recessed relative to at least a different portion of front surface 111 of platform 105. The recessed portion can be the majority of the front surface of the platform. In some embodiments, the recessed portion can be the entire front surface of the platform except for where the legs supporting the platform are located. In some embodiments, the recessed portion can be the entire front surface of the platform (when the legs holding up the platform are not included in the platform). The recessed portion in some embodiments can provide any pump heads or gas module heads located on the recessed platform access directly below the notches of the platform above it. For example, the pump heads and gas module heads can in some aspects hang off or protrude or extend over the edge of the recessed portion such that any tubing or cable through a notch in the above platform can go straight into the pump heads or gas module heads vertically without sticking off the general perimeter of the console as depicted in FIG. 2.

The recessed portion can come in a variety of shapes and sizes. In some embodiments, the recessed portion can be circular in nature (e.g., a portion of a circle), such as semicircular, or can be square, rectangular, triangular, and/or one or more of other various geometries. The recessed portion can in some embodiments have a recessed depth of about 0.5 to about 5, about 1 to about 3, about 1.5 to about 2.5, or about 2 inches into the platform. In addition, the recessed portion can have a recessed depth of about 0.5 to about 5, about 1 to about 3, about 1.5 to about 2.5, or about 2 inches, e.g., relative to at least a portion of a surface of another platform. For example, FIG. 1D is a top view of an aspect of a platform 105 that has recessed portion 115 that is shaped as a rectangle in front surface 109. In some embodiments, the recessed portion is or is approximately 12-36, 20-30, 22-24, or 23 inches along the surface. In some embodiments, the recessed portion of the surface of the platform is at least about 6 inches, at least about 12 inches, at least about 16 inches, at least about 20 inches, at least about 22 inches, or at least about 23 inches along the surface. In addition, a portion of a surface of a platform can in some aspects be recessed to be substantially even vertically or plumb with at least a portion of one or more notches of a platform above or below the recessed platform.

In some embodiments, a surface platform is not recessed and/or does not contain notches; in some aspects, such a surface of a platform can be unaltered. For example, front surface 113 and side surface 114 of platform 106 are unaltered as shown in FIG. 1E. A pump head in some aspects can be hanging off the recessed portion of a platform. In some embodiments, a platform below the recessed portion's platform in some aspects can be unaltered and/or not contain recessions or notches; in some aspects of such embodiments any tubing from the pump head can directly access a component located on the platform below the recessed portion's platform without bending or curving the tubing. For example, in some aspects, waste tube 222 can go vertically from pump module 224 on platform 205 to waste container 226 on platform 206 without sticking off the general perimeter of the console as depicted in FIG. 2.

All the surfaces of any platform described herein can in some embodiments be interchangeable. For example, the front surface, back surface, and/or side surfaces of platforms 103, 105, 106 (and any additional platforms) can have notches, be recessed, and/or be unaltered.

The front surface (and back surface) can in some embodiments be about 12 to about 36, about 20 to about 30, about 24 to about 28, or about 26 inches long. In some embodiments, the length of the front surface (and back surface) is at max 36 inches, at max 30 inches, or at max 26 inches long. A side surface can be about 25 to about 45, about 20 to about 40, about 25 to about 35, about 28 to about 32, or about 30 inches long. The thickness of a platform can be less than about 0.5 inches or about 0.5 to about 5, about 1 to about 3, about 1 to about 2, or about 1.5 inches thick. The thickness of a leg can be about 0.5 to about 5, about 1 to about 3, about 1 to about 2, or about 1.5 inches thick. The length of the platform between legs can be about 12 to about 36, about 16 to about 30, about 18 to about 26, about 20 to about 26, about 22 to about 24, or about 23 inches long. In some embodiments, the length of the platform between legs can be at least about 12 inches, at least about 16 inches, at least about 20 inches, at least about 22 inches, or at least about 23 inches long. The distance between the top surface of one platform to the bottom surface of the platform above it can be about 2 to about 12, about 4 to about 10, about 6 to about 12, or about 6 to about 8 inches. In some embodiments, the distance between the top surface of one platform to the bottom surface of the platform above it is about 12, about 10, about 8, about 6, or about 4 inches. All platforms do not have to be equally spaced apart. For example, the top surface of platform 105 can be about 8 inches below the bottom surface of platform 103 and the top surface of platform 106 can be about 6 inches below the bottom surface of platform 105. In addition, a platform can extend no more than about 2, about 4, about 6, about 8, about 10, or about 12 inches from any side of the reactor of the bioreactor (e.g., a bioreactor rocker).

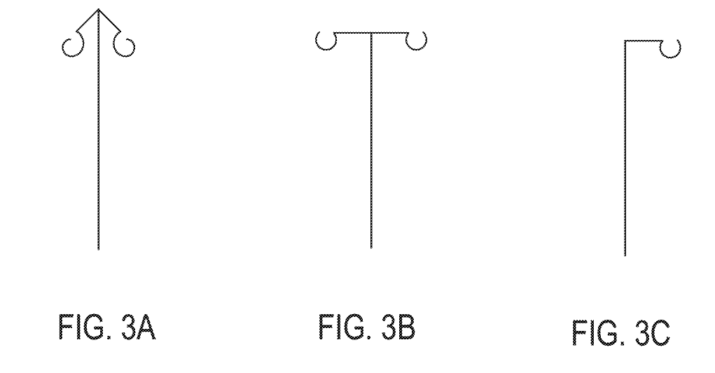

The bioreactor console can also include one or more accessory holders. In some embodiments, an accessory holder can be mounted on at least one leg of the bioreactor console, at least two legs of the bioreactor console, at least three legs of the bioreactor console, or all legs of the bioreactor console. In some embodiments, an accessory holder can be mounted to a front surface, a back surface, and/or a side surface of a platform. In some embodiments, an accessory holder can be a support for a pole such as an IV stand, a document (e.g., logbook) holder, a label holder, spare tube holder, spare cable holder, spare other part holder, or a utensil holder among others. In some embodiments, the accessory holder can include a support plate and/or a support collar. In addition, besides an IV stand, an IV support can be repurposed for other accessories including documents, cellbags, culture bags, utensils among others. In some embodiments, all four legs of the bioreactor console include an IV support or other accessory holder. An IV can hold the components or additives that go into the feed tube of the bioreactor. For example, the contents of the IV can be media and/or a cell suspension. Accordingly, an IV can either drain the contents by gravity into the bioreactor or the IV can be connected to a pump module in order to pump the contents of the IV into the bioreactor through feed tubing. FIG. 1A illustrates support plates 107 and support collars 108 which can be used to support one or multiple IV stands. For example, FIG. 2 includes removable IV stand 227 supported by support plate 207 and support collar 208 and FIGS. 4A and 4B include multiple IV stands 427 supported by support plate 407 and support collar 408. Although shown on a side surface of the leg, an accessory holder can be mounted on the front surface or back surface of a leg. In some embodiments, an IV pole is removably mounted on at least one leg of the bioreactor console. In some embodiments, a support for an IV pole can be a hole in a top surface of a platform which forms a slot for an IV pole. FIGS. 3A-3C illustrate examples of IV poles that can be used with the bioreactor console. However, additional IV poles known in the art can be used with the bioreactor console and corresponding bioreactor. In some embodiments, an accessory holder for an IV stand is designed such that the IV stand extends about 12 to about 36, about 20 to about 36, about 25 to about 35, about 24 to about 26, or about 25 inches above the top surface of the top platform.

On the bottom of each leg 101 can be feet 102. Feet 102 can be level adjusting and/or vibration dampening feet. Some bioreactors need a stable surface in order to produce accurate results during operation. For example, some bioreactors are commonly designed with integrated instruments (such as loadcells used for weight measurement) that require a stable support surface with no or limited vibration. The bioreactor consoles disclosed herein can have sufficient heft and stability to limit disturbance of the bioreactor during operation. Feet 102 not only can hold the weight of the bioreactor console and the components resting on the bioreactor console, but can provide stabilization necessary for bioreactor experiments to be conducted. For example, each foot can be adjustable to allow levelling of the bioreactor console. In some embodiments, levelling feet with locking-nuts are used on each leg. In some embodiments, vibration-dampening feet or bushings are used on each leg. In some embodiments, levelling feet with locking-nuts and vibration-dampening feet are used on each leg. The feet on the bioreactor console can be non-marking, provide good stability and vibration dampening, and/or are corrosion resistant (i.e., suitable for ISO7 cleanroom environment). The feet of the bioreactor can be made out of steel, stainless steel (e.g., 316 stainless steel), powder coated steel, ceramic-coated steel, aluminum, titanium, polymers (e.g., rubber), or combinations thereof. Additional cross-bars or other bracing may also be added to the bioreactor console for further improvement of stability as needed.

The material the bioreactor console is made out of can be steel, stainless steel (e.g., 316 stainless steel), powder coated steel, ceramic-coated steel, aluminum, titanium, or combinations thereof. In some embodiments, the platforms can be made of one material described above and the legs can be made of a different material described above. Furthermore, the bioreactor console can be cleanable and corrosion-resistant (i.e., suitable for ISO7 cleanroom environment). For example, any surface finishes and/or welds can be suitable for use in ISO7 cleanroom environment. In some embodiments, the bioreactor console without a bioreactor can weigh about 20 to about 100, about 30 to about 90, about 40 to about 80, about 50 to about 70, about 55 to about 65, or about 60 lbs. In addition, the corners and edges of the bioreactor console can be smooth and/or rounded.

The consoles disclosed herein can in some embodiments be symmetrical from front to back and/or symmetrical from side to side. Multiple bioreactor consoles in some embodiments can be set up side by side and/or front to back. In addition, the distance between consoles when two bioreactor consoles are placed side-by-side can be about 0 to about 36, about 6 to about 24, about 9 to about 16, or about 9 to about 12 inches. These distances can also be the distance between the individual bioreactors themselves. Furthermore, a bioreactor console can be stacked on top of another bioreactor console to further reduce their manufacturing footprint.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein. Finally, the entire disclosure of the patents and publications referred in this application are hereby incorporated herein by reference.

1. A console comprising:
a first platform comprising a first top surface and a first front surface, wherein the first front surface comprises one or more notches;
a second platform comprising a second top surface and a second front surface, wherein the second platform is located below the first platform and a portion of the second front surface is recessed relative to at least a portion of the first front surface;
a third platform located below the second platform; and
a plurality of legs supporting the first platform, the second platform, and/or the third platform.

2. The console of claim 1, comprising an accessory holder mounted on at least one leg of the plurality of legs.

3. The console of claim 1, further comprising a pole, which optionally is capable of securing a mounted culture bag and/or optionally is an IV pole, mounted on at least one leg of the plurality of legs.

4. The console of claim 1, wherein the plurality of legs comprises four legs and accessory holders are mounted on at least two of the four legs.

5. The console of claim 1, wherein the one or more notches comprised by the first front surface comprises three notches.

6. The console of claim 1, wherein one or more of the one or more notches are semicircular.

7. The console of claim 1, where the first platform comprises a first back surface and optionally wherein the first back surface comprises one or more notches.

8. The console of claim 6, wherein a diameter of the one or more notches is 1 to 6 inches.

9. The console of claim 1, wherein the second front surface is recessed to be substantially even vertically or plumb with at least a portion of the one or more notches.

10. The console of claim 1, wherein the first top surface is between 2 and 4 feet from a bottom surface of at least one leg of the plurality of legs.

11. The console of claim 1, wherein the first front surface is between 20 and 40 inches long.

12. The console of claim 1, wherein the first platform comprises a first side surface that is between 25 and 45 inches long.

13. The console of claim 1, wherein the first platform, the second platform and the third platform comprise stainless steel.

14. The console of claim 1, wherein each leg of the plurality of legs comprises a vibration dampening foot.

15. The console of claim 14, each vibration dampening foot is adjustable, optionally to allow levelling of the bioreactor console.

16. The console of claim 1, wherein the first platform comprises a first bottom surface and the second top surface is 6 to 12 inches below the first bottom surface.

17. The console of claim 1, wherein the second platform comprises a second bottom surface and the third top surface is 4 to 10 inches below the second bottom surface.

18. The console of claim 1, wherein the first top surface comprises a plurality of tubing clips positioned to guide a plurality of tubes to the one or more notches.

19. The console of claim 1, wherein the first top surface comprises plurality of tubing clips and a plurality of color coded markers positioned to guide a tube to each of the plurality of notches.

20. A bioreactor system comprising:
a bioreactor rocker supported on a first platform, wherein the first platform comprises a first top surface and a first front surface comprising one or more notches;
a bioreactor pump module supported on a second platform located below the first platform, wherein the second platform comprises a second top surface and a second front surface, and wherein a portion of the second front surface is recessed relative to at least a portion of the first front surface;
a third platform located below the second platform; and
a plurality of legs supporting the first platform, the second platform and/or the third platform.

21. The bioreactor system of claim 20, comprising a waste container supported on the third platform.

22. The bioreactor system of claim 20, comprising a bioreactor gas module supported on the second platform.

23. The bioreactor system of claim 20, wherein the first platform extends no more than 12 inches from any side of the bioreactor rocker.

24. The bioreactor system of claim 20, comprising an accessory holder mounted on at least one leg of the plurality of legs.

25. The bioreactor system of claim 20, comprising an IV pole mounted on at least one leg of the plurality of legs.

26. The bioreactor system of claim 20, wherein the bioreactor system comprises four legs and accessory holders are mounted on at least two of the four legs.

27. The bioreactor system of claim 20, wherein the first front surface comprises three notches.

28. The bioreactor system of claim 20, wherein the one or more notches are semicircular.

29. The bioreactor system of claim 20, where the first platform comprises a first back surface and the first back surface comprises one or more notches.

30. The bioreactor system of claim 28, wherein a diameter of the one or more notches is 1 to 6 inches.

31. The bioreactor system of claim 20, wherein the second front surface is recessed to be substantially even vertically or plumb with at least a portion of the one or more notches.

32. The bioreactor system of claim 20, wherein the first top surface is between 2 and 4 feet from a bottom surface of at least one leg of the plurality of legs.

33. The bioreactor system of claim 20, wherein the first front surface is between 20 and 40 inches long.

34. The bioreactor system of claim 20, wherein the first platform comprises a first side surface that is between 25 and 45 inches long.

35. The bioreactor system of claim 20, wherein the first platform, the second platform and the third platform comprise stainless steel.

36. The bioreactor system of claim 20, wherein each leg of the plurality of legs comprises a vibration dampening foot.

37. The bioreactor system of claim 36, each vibration dampening foot is adjustable to allow levelling of the bioreactor system.

38. The bioreactor system of claim 20, wherein the first platform comprises a first bottom surface and the second top surface is 6 to 12 inches below the first bottom surface.

39. The bioreactor system of claim 20, wherein the second platform comprises a second bottom surface and the third top surface is 4 to 10 inches below the second bottom surface.

40. The bioreactor system of claim 20, wherein the first top surface comprises a plurality of tubing clips positioned to guide a plurality of tubes from the bioreactor rocker to the one or more notches.

41. The bioreactor system of claim 20, wherein the first top surface comprises plurality of notches and a plurality of color coded markers positioned to guide a tube to each of the plurality of notches.

42. A bioreactor system comprising:
a bioreactor rocker supported on a first platform, wherein the first platform comprises a first top surface and a first front surface comprising one or more notches;
a bioreactor pump module supported on a second platform located below the first platform, wherein the second platform comprises a second top surface and a second front surface, and wherein the second front surface is recessed relative to at least a portion of the first front surface;
a bioreactor gas module supported on the second platform;
a waste container supported on a third platform located below the second platform;
a plurality of legs supporting the first platform, the second platform and/or the third platform; and
an IV pole mounted on at least one leg of the plurality of legs.
